(19) United States
(12) Patent Application Publication
(10) Pub. No.: US 2019/0169565 A1
(43) Pub. Date: Jun. 6, 2019

(54) MEDIUM AND METHOD FOR CULTURING ANTRODIA CINNAMOMEA

(71) Applicant: I-SHOU UNIVERSITY, Kaohsiung (TW)

(72) Inventors: Chih-Hui YANG, Kaohsiung (TW); Keng-Shiang Huang, Kaohsiung (TW); Cheng-Ming Tsai, Kaohsiung (TW); Yu-Mei Lin, Kaohsiung (TW)

(21) Appl. No.: 16/054,384

(22) Filed: Aug. 3, 2018

(30) Foreign Application Priority Data
Dec. 1, 2017 (TW) 106142249

(51) Int. Cl. C12N 1/14

(52) U.S. Cl. CPC C12N 1/14; A01H 15/00

(57) ABSTRACT

A medium for culturing Antrodia cinnamomea includes a carbon source, a nitrogen source, vitamin A, agar and a solvent. The medium includes 8-16 grams/liter of the carbon source, 0.4-1.6 grams/liter of the nitrogen source and 4 grams/liter of vitamin A. Moreover, a method for culturing Antrodia cinnamomea includes inoculating the medium with mycelia of Antrodia cinnamomea. The mycelia of Antrodia cinnamomea inoculated on the medium is cultured at 20-27° C. for 28-50 days.

### CROSS REFERENCE TO RELATED APPLICATIONS

The application claims the benefit of Taiwan application serial No. 106142249, filed Dec. 1, 2017, the entire contents of which are incorporated herein by reference.

### BACKGROUND OF THE INVENTION

### 1. Field of the Invention

The present invention generally relates to a medium and, more particularly, to a medium for culturing Antrodia cinnamomea. The present invention also relates to a method for culturing Antrodia cinnamomea using the medium.

### 2. Description of the Related Art

Antrodia cinnamomea, a precious traditional Chinese medicine in Taiwan, grows only on inner rotten walls of hollow materials from a conserving species of Cinnamomum kanehirai. Wild Antrodia cinnamomea is rich in physiologically active ingredients such as triterpenoids and polysaccharides. Triterpenoids possess effects such as anti-tumor, liver-protective, anti-dotal, anti-high blood lipid and pressure and immuno-modulating activities. Polysaccharides show effect on stimulation of macrophages, improvement of immune-regulation, clearance of free radicals and anti-hepatitis B virus. As a result, wild Antrodia cinnamomea becomes precious and expensive.

Due to several problems of wild Antrodia cinnamomea, such as rare amount and difficulty in obtaining, industries tend to culture Antrodia cinnamomea by conventional methods and produce mycelia or fruit bodies of Antrodia cinnamomea. Furthermore, physiologically active ingredients, such as triterpenes and polysaccharides, can be further extracted from the mycelia and fruit bodies of Antrodia cinnamomea.

As an example, solid state fermentation is one of the conventional method for culturing mycelia of Antrodia cinnamomea, using a conventional medium including substances such as wood flour, soybean powders, calcium carbonate (CaCO₃), glucose, etc. The solid state fermentation is wildly used in the industry due to the following reasons. First, the mycelia of Antrodia cinnamomea with similar physiologically active ingredients can be obtained due to the substances of the conventional medium. Second, the solid state fermentation is easy to operate. Third, the substances of the conventional medium are easily obtained. Fourth, the conventional medium used in the solid state should be kept in low water content, and thus is not easy to be used by other microorganisms. However, color of the mycelia of Antrodia cinnamomea obtained by the solid state fermentation is not red enough, and thus decreases the consumer's willingness to purchase the mycelia of Antrodia cinnamomea obtained by the solid state fermentation. In light of this, the conventional medium should be improved.

### SUMMARY OF THE INVENTION

It is therefore an objective of the present invention to provide a medium for culturing Antrodia cinnamomea, which is used to obtain mycelia of Antrodia cinnamomea in red color.

It is therefore another objective of the present invention to provide a method for culturing Antrodia cinnamomea to culture the mycelia of Antrodia cinnamomea by the medium.

One embodiment of the invention discloses the medium for culturing Antrodia cinnamomea includes a carbon source, a nitrogen source, vitamin A, agar and a solvent. The medium includes 8-16 grams/liter of the carbon source, 0.4-1.6 grams/liter of the nitrogen source and 4 grams/liter of vitamin A. Thus, mycelia of Antrodia cinnamomea grown on the medium can uptake vitamin A in the medium and the mycelia of Antrodia cinnamomea in red color can be obtained.

In an example, the carbon source is selected from the group consisting of malt extract, glucose, fructose, galactose, lactose, starch, cellulose, sucrose and combination therof. The nitrogen source is selected from the group consisting of peptone, yeast extract, whey protein, tryptone, amino acids, urea, ammonium acetate, ammonium chloride, ammonium nitrate, potassium nitrate, sodium nitrate and combination therof.

In an example, the medium can include 0.8% (w/v) of malt extract, 0.8% (w/v) of glucose and 0.04% (w/v) of peptone. Alternatively, the medium can include 0.8% (w/v) of malt extract, 0.08% (w/v) of peptone and 0.08% (w/v) of yeast extract.

Another embodiment of the invention discloses the method for culturing Antrodia cinnamomea. The method includes inoculating a medium with mycelia of Antrodia cinnamomea. The mycelia of Antrodia cinnamomea inoculated on the medium is then cultured at 20-27° C. for 28-50 days. Thus, mycelia of Antrodia cinnamomea grown on the medium can uptake vitamin A in the medium and the mycelia of Antrodia cinnamomea in red color can be obtained.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 depicts a chemical structure of vitamin A.

FIG. 2a depicts photographs illustrating growth status of mycelia of Antrodia cinnamomea grown on medium according to a first embodiment of the present invention.

FIG. 2b depicts a bar chart illustrating average diameter of the mycelia of Antrodia cinnamomea grown on medium according to the first embodiment of the present invention.

FIG. 3a depicts photographs illustrating growth status of mycelia of Antrodia cinnamomea grown on medium according to a second embodiment of the present invention.

FIG. 3b depicts a bar chart illustrating average diameter of the mycelia of Antrodia cinnamomea grown on medium according to the second embodiment of the present invention.

### DETAILED DESCRIPTION OF THE INVENTION

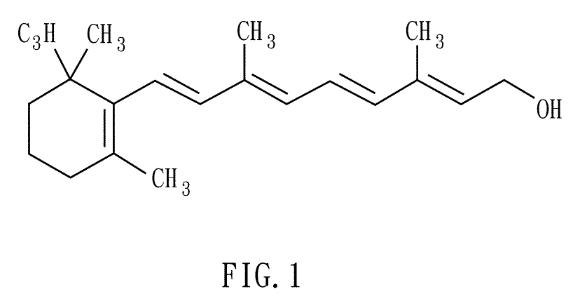

Vitamin A with a chemical structure shown in FIG. 1 is one of the fat-soluble vitamins. Vitamin A does not easily lose due to metabolism, and can be kept in cells, which is appreciated by a person having ordinary skill in the art. Detail description is not given to avoid redundancy.

Specifically, vitamin A can be used as an additive in a medium for culturing Antrodia cinnamomea. That is, 1 liter of the medium includes 4 grams of vitamin A. Therefore, when the medium is used to culture mycelia of Antrodia cinnamomea, as an example, Antrodia cinnamomea strains with BCRC number of BCRC 35396, BCRC 35716, BCRC 36401 and BCRC 37848, vitamin A in the medium can be uptaken by the mycelia of Antrodia cinnamomea and the mycelia of Antrodia cinnamomea in red color can thus be obtained.

Moreover, the medium can further include a carbon source as well as a nitrogen source for growth of Antrodia cinnamomea. As an example, the medium can include 0.8-1.6% (w/v) of a carbon source and 0.04-0.16% (w/v) of a nitrogen source. That is, 8-16 grams of the carbon source, 0.4-1.6 grams of the nitrogen source and 4 grams of vitamin A are mixed, and a solvent, such as distilled water, is added until the total volume is 1 liter. The carbon source can be selected from the group consisting of malt extract, glucose, fructose, galactose, lactose, starch, cellulose, sucrose and combination therof. The nitrogen source can be selected from the group consisting of peptone, yeast extract, whey protein, tryptone, amino acids, urea, ammonium acetate (CH₃COONH₄), ammonium chloride (NH₄Cl), ammonium nitrate (NH₄NO₃), potassium nitrate (KNO₃), sodium nitrate (NaNO₃) and combination therof. In addition, the medium can also include proper amount of agar, and thus the medium can be formed in a solid state and can be used in the solid state fermentation.

The medium according to a first embodiment of the present invention includes 0.8% (w/v) of malt extract, 0.8% (w/v) of glucose, 0.04% (w/v) of peptone, 0.4% (w/v) of vitamin A, 0.8% (w/v) of agar and 97.16% (w/v) of water. Besides, the medium according to a second embodiment of the present invention includes 0.8% (w/v) of malt extract, 0.08% (w/v) of peptone, 0.08% (w/v) of yeast extract, 0.4% (w/v) of vitamin A, 0.8% (w/v) of agar and 97.84% (w/v) of water.

Therefore, the medium according to the first and the second embodiments of the present invention can be applied in a method for culturing Antrodia cinnamomea. The medium is inoculated with the mycelia of Antrodia cinnamomea, and the mycelia of Antrodia cinnamomea inoculated on the medium is cultured at 20-27° C. for 28-50 days. The mycelia of Antrodia cinnamomea in red color can thus be obtained.

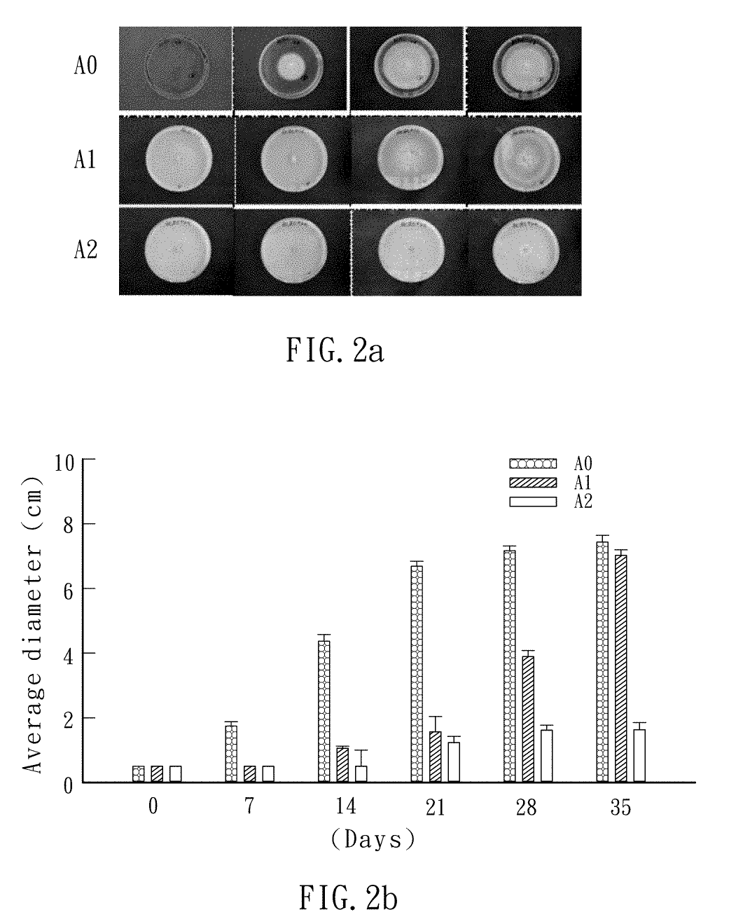

To evaluate the medium with vitamin A as the additive can be used to obtain the mycelia of Antrodia cinnamomea in red color, the mycelia of Antrodia cinnamomea is inoculated on the medium listed in TABLE 1, and cultured at 20-27° C. for 35 days. Growth status and average diameter of colonies on the medium on days 0, 7, 14, 28 and 35 are shown in FIGS. 2a and 2b.

TABLE 1

| | Medium | | | | | |
|---|---|---|---|---|---|---|
| | Malt | | | Vitamin | Growth status | |
| | extract | Glucose | Peptone | A | Biomass | Growth rate |
| Group | (%) | (%) | (%) | (%) | (mg) | (R²) |
| A0 | 0.8 | 0.8 | 0.04 | 0 | 3.4 | 0.2174 |
| | | | | | | R² = 0.9598 |
| A1 | 0.8 | 0.8 | 0.04 | 0.4 | 6.85 | 0.1767 |
| | | | | | | R² = 0.8968 |
| A2 | 0.8 | 0.8 | 0.04 | 0.8 | 4.49 | 0.0398 |
| | | | | | | R² = 0.9251 |

Referring to FIG. 2a, the mycelia of Antrodia cinnamomea obtained by using the medium of groups A0-A2 have color with hexadecimal color codes of #FFC78E, #FF5809 and #FFEEDD, respectively. That is, the mycelia of Antrodia cinnamomea obtained by using the medium of group A1 (with vitamin A in the concentration of 0.4% (w/v)) is redder than the mycelia of Antrodia cinnamomea obtained by using either the medium of group A0 (without vitamin A) or the medium of group A2 (with vitamin A in a concentration of 0.8% (w/v)). Moreover, referring to TABLE 1, on day 35, biomass of the mycelia of Antrodia cinnamomea obtained by using the medium of group A1 significantly increases compared to biomass of the mycelia of Antrodia cinnamomea obtained by using the medium of group A0 or A2. Referring to FIG. 2b, the mycelia of Antrodia cinnamomea obtained by using the medium of group A1 has similar diameter with the mycelia of Antrodia cinnamomea obtained by using the medium of group A0.

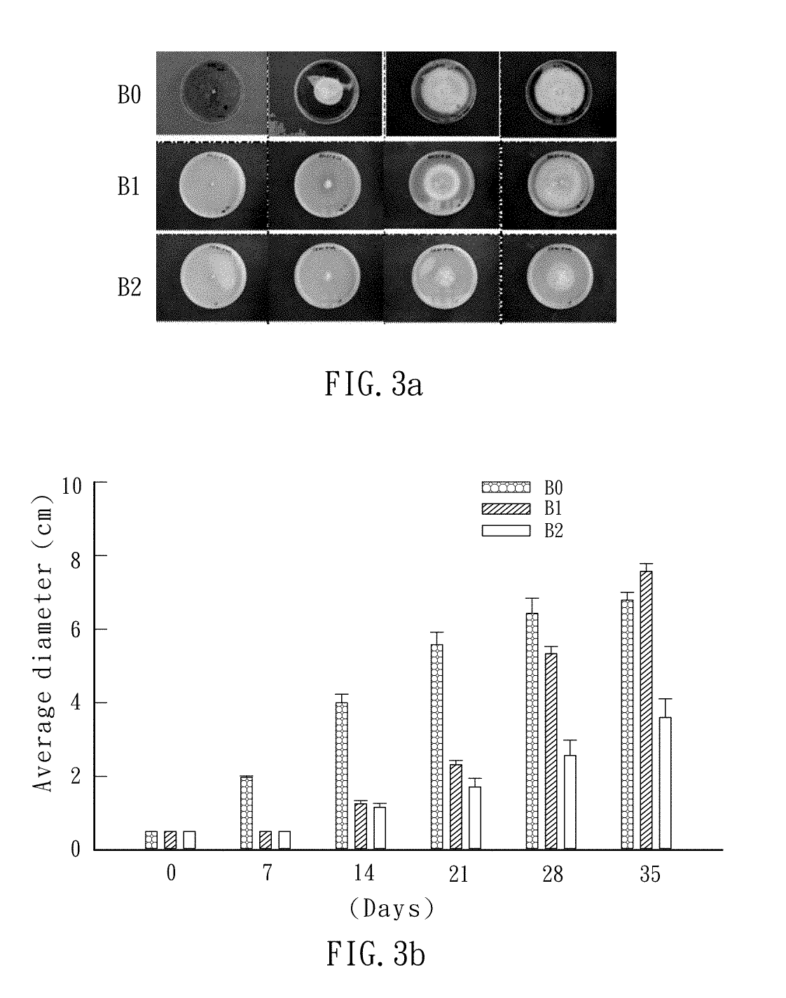

In addition, the mycelia of Antrodia cinnamomea is inoculated on the medium listed in TABLE 2, and cultured at 20-27° C. for 35 days. Growth status and average diameter of colonies on the medium on days 0, 7, 14, 28 and 35 are shown in FIGS. 3a and 3b.

TABLE 2

| | Medium | | | | | |
|---|---|---|---|---|---|---|
| | Malt | | | Vitamin | Growth status | |
| | extract | Glucose | Peptone | A | Biomass | Growth rate |
| Group | (%) | (%) | (%) | (%) | (mg) | (R²) |
| B0 | 0.8 | 0.08 | 0.08 | 0 | 2.60 | 0.1893 |
| | | | | | | R² = 0.9763 |
| B1 | 0.8 | 0.08 | 0.08 | 0.4 | 4.35 | 0.2078 |
| | | | | | | R² = 0.9356 |
| B2 | 0.8 | 0.08 | 0.08 | 0.8 | 5.40 | 0.0908 |
| | | | | | | R² = 0.9693 |

Referring to FIG. 3a, the mycelia of Antrodia cinnamomea obtained by using the medium of groups B0-B2 have color with hexadecimal color codes of #FFD1A4, #FF8040 and #FFEEDD, respectively. That is, the mycelia of Antrodia cinnamomea obtained by using the medium of group B1 (with vitamin A in the concentration of 0.4% (w/v)) is redder than the mycelia of Antrodia cinnamomea obtained by using either the medium of group B0 (without vitamin A) or the medium of group B2 (with vitamin A in a concentration of 0.8% (w/v)). Moreover, referring to TABLE 2, on day 35, biomass of the mycelia of Antrodia cinnamomea obtained by using the medium of group B1 significantly increases compared to biomass of the mycelia of Antrodia cinnamomea obtained by using the medium of group B0 or B2. Referring to FIG. 3b, the mycelia of Antrodia cinnamomea obtained by using the medium of group B1 has similar diameter with the mycelia of Antrodia cinnamomea obtained by using the medium of group B0.

Accordingly, the mycelia of Antrodia cinnamomea grown on the medium can uptake vitamin A in the medium and the mycelia of Antrodia cinnamomea in red color can be obtained. Therefore, the consumer's willingness to purchase the mycelia of Antrodia cinnamomea increases.

Although the invention has been described in detail with reference to its presently preferable embodiment, it will be understood by one of ordinary skill in the art that various modifications can be made without departing from the spirit and the scope of the invention, as set forth in the appended claims.

What is claimed is:

1. A medium for culturing Antrodia cinnamomea, comprising:
0.8-1.6% (w/v) of a carbon source;
0.04-0.16% (w/v) of a nitrogen source;
0.4% (w/v) of vitamin A;
agar; and
a solvent until 100% (w/v).

2. The medium as claimed in claim 1, wherein the carbon source is selected from the group consisting of malt extract, glucose, fructose, galactose, lactose, starch, cellulose, sucrose and combination therof.

3. The medium as claimed in claim 1, wherein the nitrogen source is selected from the group consisting of peptone, yeast extract, whey protein, tryptone, amino acids, urea, ammonium acetate, ammonium chloride, ammonium nitrate, potassium nitrate, sodium nitrate and combination therof.

4. The medium as claimed in claim 1, wherein the medium comprises 0.8% (w/v) of malt extract, 0.8% (w/v) of glucose and 0.04% (w/v) of peptone.

5. The medium as claimed in claim 1, wherein the medium comprises 0.8% (w/v) of malt extract, 0.08% (w/v) of peptone and 0.08% (w/v) of yeast extract.

6. A method for culturing Antrodia cinnamomea, comprising:
inoculating a medium with mycelia of Antrodia cinnamomea, wherein the medium comprises: 0.8-1.6% (w/v) of a carbon source, 0.04-0.16% (w/v) of a nitrogen source, 0.4% (w/v) of vitamin A, agar and a solvent until 100% (w/v); and
culturing the mycelia of Antrodia cinnamomea inoculated on the medium at 20-27° C. for 28-50 days.
